(19) United States
(12) Patent Application Publication
(10) Pub. No.: US 2010/0098924 A1
(43) Pub. Date: Apr. 22, 2010

(54) Collagen membrane for medical use and method for manufacturing the same

(75) Inventors: Tzu-Yin Hsiao, Taipei City (TW); Yu-Te Lin, Anding Township (TW); Chien-Hsing Lin, Rende Township (TW); Hsiang-Yin Lu, Pingtung City (TW)

Correspondence Address: BACON & THOMAS, PLLC, 625 SLATERS LANE, FOURTH FLOOR, ALEXANDRIA, VA, 22314-1176, US

(73) Assignee: SunMax Biotechnology Co., Ltd., Tainan County (TW)

(21) Appl. No.: 12/379,006

(22) Filed: Feb. 11, 2009

(30) Foreign Application Priority Data
Oct. 16, 2008 (TW) 097139723

(51) Int. Cl. C07K 14/78

(52) U.S. Cl. 428/219; 530/427

(57) ABSTRACT

The present invention relates to a method for manufacturing a collagen membrane, which comprises the following steps: preparing a collagen slurry; degassing the collagen slurry, and then forming collagen gel at a predetermined collagen concentration, ionic strength, pH value, and temperature; removing water in the collagen gel by an absorbent device to form a collagen mat; and flattening and drying the collagen mat under vacuum by a gel dryer. The present invention provides the collagen membranes manufactured by the above-mentioned method. Such membranes are of good biocompatibility, high tensile strength, hydrophilicity, flexibility and easy handling features. Hence, these membranes can function as medical material suitable for repair of tissue and wound healing.

### BACKGROUND OF THE INVENTION

1. Field of the Invention

The present invention relates to a collagen membrane and a method for manufacturing the same and, more particularly, to a collagen membrane suitable for medical applications in repair of tissue and wound healing and a method for manufacturing the same.

2. Description of Related Art

Currently, considerable research has been made by various groups to enable artificial membranes to substitute skin, blood vessels, ligaments, and other connective tissues. Since collagen is a major component of connective tissues, it is generally used as a material of these artificial membranes.

Nevertheless, such artificial membranes have properties affected not only by the nature of collagen used therein, but also by the process of the manufacture thereof. Many have less than optimal properties of flexibility, biological stability, strength, and ease of handling. To achieve desired membrane properties suitable for uses in a variety of medical applications including periodontal regeneration, dura mater repair, cartilage repair, tendon replacement, tissue reinforcement, anti-adhesion, etc, appropriate selection from a spectrum of processes must be determined.

### SUMMARY OF THE INVENTION

The object of the present invention is to provide a collagen membrane and a method for manufacturing the same. The collagen membrane can be obtained by the method of the present invention. This collagen membrane possesses biodegradability, good biocompatibility, high tensile strength, hydrophilicity, flexibility, and easy handling features. Its compact structure can function as a barrier in medical applications to accelerate healing of tissue.

To achieve the object, the method of the present invention includes the following steps: preparing a collagen slurry; degassing the collagen slurry, and then forming collagen gel at predetermined collagen concentration, ionic strength, pH value, and temperature; removing water in the collagen gel by an absorbent device to form a collagen mat; and flattening and drying the collagen mat under vacuum by a gel dryer.

The resulting membrane of the present invention is a translucent, smooth, and flexible film with relatively high tensile strength when the membrane is either dry or wet. The properties of the membrane may be further modified by mixing cross-linked collagen fibril in the collagen slurry.

In the foregoing method of the present invention, collagen used in the collagen slurry is prepared from soluble collagen monomer dissolved in HCl, pH 2˜3 (not being formed into fibril, i.e. it is purified atelopeptide collagen with a native triple-helical structure of molecular weight of around 300 kD), or the combination of the above-mentioned soluble collagen monomer solution and cross-linked collagen fibril paste. In the abovementioned combination, the ratio of the collagen mass of the soluble collagen monomer solution to the cross-linked collagen fibril paste is preferably in the range of 9.5:0.5 to 8:2. Commonly, the cross-linked collagen fibril can be prepared from the soluble collagen monomer by fibril reconstitution process and then treated with a cross-linking agent which can be conventional, for example, formaldehyde, glutaraldehyde, glyoxal, pyruvic aldehyde, aldehyde starch, and so on, but preferably is glutaraldehyde.

Furthermore, the collagen concentration in the collagen slurry can be in a range of 1 to 5 mg/mL, but preferably 3 mg/mL. Nine volumes of the collagen slurry are mixed with one volume of a phosphate buffer (pH 10˜12 and 0.1˜0.3 M) to bring the slurry to physiological pH and ionic strength. The collagen slurry starts to form collagen gel by incubation at 20˜40° C. under no agitation condition for overnight. Preferably, the phosphate buffer is of pH 11±0.2 and concentration in 0.2 M. NaCL can be added to slow the gelling rate. The incubation temperature is 30±5° C. The degassing process under vacuum is required before gel formation. The vacuum level of the degassing process can range from −400 to −700 mmHg, and preferably is from −500 to 650 mmHg. In addition, the final flattening and drying by the gel dryer can be at a heating temperature of 45±5° C. under vacuum.

Moreover, the absorbent device can comprise an absorbent material, a porous net covering the absorbent material, and a housing receiving the absorbent material and the porous net. The absorbent material can be a sterile non-woven fabric pad, a sterile cotton pad, or the combination thereof. The porous net is a sterile nylon net, a sterile metal net, or the combination thereof, and preferably is of pore size ranging from 100 to 150 μm. The housing of the absorbent device mentioned above can be made of conventional stainless steel.

The present invention also provides collagen membranes prepared according to the aforesaid method. The density of these collagen membranes can be in the range of 0.1 to 2 g/cm³.

Other objects, advantages, and novel features of the invention will become more apparent from the following detailed description when taken in conjunction with the accompanying drawings.

### BRIEF DESCRIPTION OF THE DRAWINGS

FIG. 1 shows a perspective view of a gel mold used in Example 1 of the present invention;

FIG. 2a shows a breakdown view of an absorbent device used in Example 1 of the present invention;

FIG. 2b shows a perspective view of the absorbent device used in Example 1 of the present invention; and

FIG. 3 shows a gel dryer used in Example 1 of the present invention.

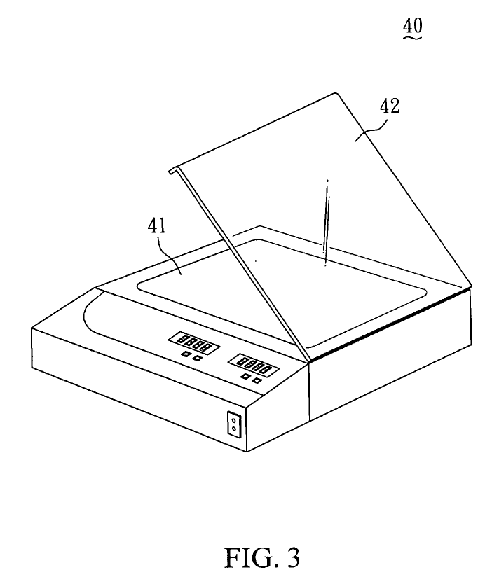

### DETAILED DESCRIPTION OF THE PREFERRED EMBODIMENT

Because of the specific embodiments illustrating the practice of the present invention, a person having ordinary skill in the art can easily understand other advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

Preparation of Cross-linked Collagen Fibril

First, soluble collagen monomers (3 mg/ml, pH 2.0 in 0.01 N HCl, prepared with reference to TW Patent No. 1236501) and a phosphate buffer were mixed in a ratio of 9:1 by weight or volume, in which the phosphate buffer is of pH 11.0±0.2 and concentration in 0.2 M. During the process of the mixing, the collagen solution was continuously stirred, adjusted the pH value to be 7.0±0.2 and then consecutively reacted for 4 hours at 30±5° C. As a result, collagen therein can be reconstituted into collagen fibril (uncross-linked).

The collagen fibril solution, as prepared above was added with a cross-linking agent, 0.005% glutaraldehyde, followed by being reacted for 16 hours at 30±5° C. Then, cross-linked collagen fibril was collected from the collagen fibril solution by centrifuging under 14,000 G for 1 hour. In general, the concentration of the cross-linked collagen fibril paste made according to the steps mentioned above can range from 65.0 to 100.0 mg/mL. In addition to glutaraldehyde, other cross-linking agents such as formaldehyde, glyoxal, pyruvic aldehyde, and aldehyde starch can be used.

The cross-linked collagen fibril paste was washed with a phosphate buffer (100 mL, pH 7.0±0.2, 0.02 M) three times, and then its collagen concentration was diluted to 35±2.0 mg/mL therewith.

### EXAMPLE 1

The soluble collagen monomer solution (300 mL, 3±0.3 mg/mL) and the cross-linked collagen fibril paste (2.86 mL, 35±0.2 mg/mL) were mixed by stirring for 10 minutes, resulting in both of them uniformly dispersing in this mixed slurry. The ratio of the collagen mass of the soluble collagen monomer solution to the cross-linked collagen fibril paste was in the range of 9.5:0.5 to 8:2. Besides, the soluble collagen monomer solution can be used alone to prepare the collagen slurry without addition of the cross-linked collagen fibril paste.

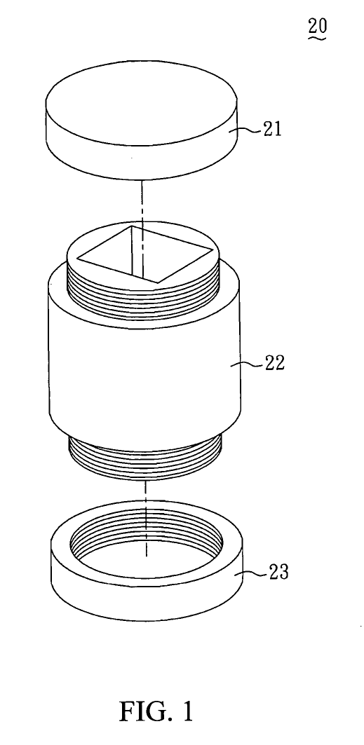

Nine volumes of this mixed slurry (containing the soluble collagen monomer and the cross-linked collagen fibril) were mixed with one volume of a phosphate buffer (33.3 mL, 0.2 M, pH 11.0±0.2, NaCl 1˜2 M). The phosphate buffer was not limited to pH 11.0±0.2 and concentration in 0.2 M. Any other pH value and concentration can be chosen as long as the pH value is in the range of 10 to 12 and the concentration is in the range of 0.1 to 0.3 M. The pH of the mixture was then adjusted to be neutral. Subsequently, the mixed slurry was poured into a mold 20 (as shown in FIG. 1) which can be made of any conventional stainless steel. The mold was hollow and included a top 21, a hollow column 22 having a cubic space in the center, and a bottom 23. Prior to the mold 20 being used, the bottom 23 and the hollow column 22 were tightly screwed with each other, and then the mixed slurry was poured thereinto. Subsequently, the mixed slurry was degassed for 10 minutes under −400 to −600 mmHg, followed by screwing the top 21 on the hollow column 22, and then stood for overnight at incubation temperature (30±5° C.) without any agitation or shaking to form gel from the slurry.

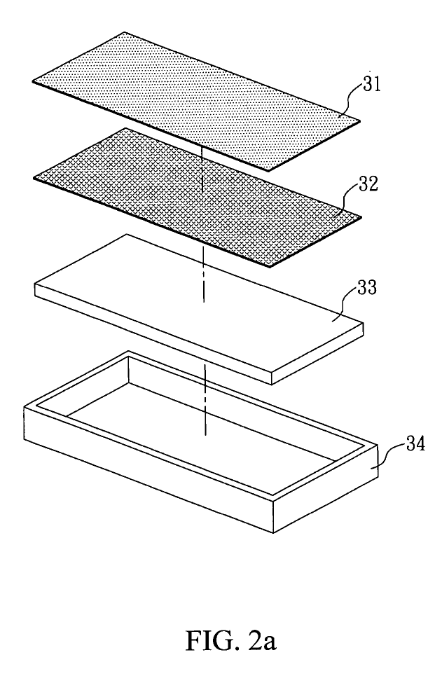

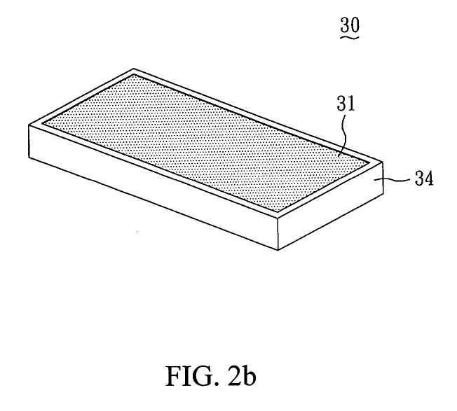

After the collagen gel was formed, the top 21 was removed and an absorbent device 30 (see FIG. 2b) was put on the mold 20. Then, the absorbent device 30 and the mold 20 were placed upside down to allow the gel in the mold 20 falling slowly on the absorbent device 30, leading to the water in the collagen gel being removed. The absorbent device 30 included four sterile members, as shown in FIG. 2a, which respectively were nylon or metal net 31 as a first film, a non-woven fabric film 32 as a second film, an absorbent cotton pad 33 as a third film, and a device housing 34. The pore size of the nylon or metal net 31 can range from 80 to 125 μm. The metal net and the device housing 34 can be made of stainless steel. With reference to FIG. 2a, the absorbent cotton material 33, the non-woven fabric film 32, and the nylon or metal net 31 were sequentially put in the device housing 34 so as to assemble the absorbent device 30. The rate of water in the collagen gel absorbed by the absorbent device 30 can achieve 5 mL/min.

After the water in the absorbent device 30 reached saturation, the absorbent device 30 was removed. The top 21 was screwed on the mold 20, and then the mold 20 was inverted. The bottom 23 was removed and another absorbent device 30 was put on the mold 20. Subsequently, the mold 20 and the absorbent device 30 were placed upside down again. Hence, the water in the collagen gel can be removed at both terminals thereof by consecutively changing the absorbent device 30. The above-mentioned steps were repeated until the collagen gel was formed into a mat of the thickness about 0.5 cm.

A sterile nylon net (pore size: 80 to 125 μm) was put on heating plate 41 of a gel dryer 40. The collagen mat was sit on the nylon net, covered with another sterile nylon net (pore size: 80 to 125 μm) on the top, and flattened by a top 42 of the gel dryer 40 under vacuum-heating condition at −650 mmHg and 45±5° C. for 1 to 3 hours until the collagen mat was formed into a collagen membrane of the thickness about 0.01 cm. This collagen membrane has the density about 1.0±0.2 g/cm³ and moisture content less than 16%. The collagen membrane was semitransparent in smooth and flat appearance. Finally, the collagen membrane could be trimmed to desired dimensions, packaged and then sterilized by radiation.

### EXAMPLE 2

Except that the collagen membrane was made of the soluble collagen monomer solution only, without addition of any cross-linked collagen fibril, the collagen membrane of the present example was prepared in the manner substantially similar to Example 1. The resulting membrane has less tensile strength and shorter biodegradation time, compared with those of collagen membrane of Example 1.

### Comparative Example

PeriAid® (produced from Collagen Matrix, Inc., U.S.A.) was used as a comparative material. It's indicated for use in patients with moderate to severe periodontal disease as a resorbable material for placement in periodontal defects to aid in wound healing post periodontal surgery.

### Experimental Example Properties of the Collagen Membranes

### I. Density

First, the length, width, and thickness of the collagen membranes prepared in the aforesaid examples were measured for calculation of the volume thereof. These collagen membranes were weighed by an electronic balance in order to calculate their density. The final results are listed in the following Table 1.

TABLE 1

| Collagen | Length × Width × Thickness | Volume | Weight | Density |
|---|---|---|---|---|
| Membrane | (cm) | (cm³) | (g) | (g/cm³) |
| Comparative | 1.5 * 1.5 * 0.025 | 0.056 | 0.0254 | 0.45 |
| Example | | | | |
| Example 1 | 1.5 * 1.5 * 0.01 | 0.0225 | 0.0270 | 1.2 |

It can be known from Table 1 that the collagen membrane prepared in Example 1 of the present invention is thinner than that of Comparative Example. As a result, the collagen membrane prepared in Example 1 of the present invention possesses smaller volume and higher density than that of Comparative Example.

### II. Tensile Strength

The collagen membranes prepared in Examples 1 and 2 were cut into pieces of 30×70 mm². The cut pieces were immersed in PBS (pH 7.0 around) at 25° C. for 10 minutes.

Each of the respective immersed pieces was clamped by a tensile test device at 20 mm distinct from the boundaries of the contrary ends. The tensile force (30 mm/min) of the test device was continuously applied on the clamped piece until the piece of the collagen membrane was torn. Finally, the tensile strength was obtained. The test results are shown in the subsequent Table 2.

TABLE 2

| Tensile | | | | |
|---|---|---|---|---|
| Strength (Mpa) | Test 1 | Test 2 | Test 3 | Average |
| Example 2 | 1.64 | 2.18 | 1.86 | 1.89 |
| Example 1 | 10.3 | 10.2 | 8.50 | 9.67 |

As shown in Table 2, addition of the cross-linked collagen fibril can significantly increase the tensile strength of the collagen membrane. Whether the cross-linked collagen fibril was added or not and its addition ratio both depend on the properties desired by different medical applications.

### III. Observation by Scanning Electron Microscope

The surface and cross-section of the collagen membrane prepared in Example 1 of the present invention were observed by a scanning electron microscope. The results showed the collagen membrane prepared in Example 1 of the present invention. It had a compact structure as seen on the cross-section and surface SEM photos.

The present invention uses the mixed slurry (containing the soluble collagen monomer solution and the cross-linked collagen fibril paste) along with gel formation and gel vacuum-drying to obtain the collagen membranes having flat surface, uniform thickness, high tensile strength, hydrophilicity, and compact structure capable of functioning as a barrier. Hence, the collagen membranes of the present invention can serve for medical applications in periodontal regeneration, dura mater repair, cartilage repair, tendon replacement, tissue reinforcement, anti-adhesion, etc.

Although the present invention has been explained in relation to its preferred embodiment, it is to be understood that many other possible modifications and variations can be made without departing from the scope of the invention as hereinafter claimed.

What is claimed is:

1. A method for manufacturing a collagen membrane comprising the following steps:
preparing a collagen slurry;
degassing the collagen slurry, and then forming collagen gel at predetermined collagen concentration, ionic strength, pH value, and temperature;
removing water in the collagen gel by an absorbent device to form a collagen mat; and
flattening and drying the collagen mat under vacuum by a gel dryer.

2. The method as claimed in claim 1, wherein collagen used in the collagen slurry is a soluble collagen monomer solution, or the combination of the soluble collagen monomer solution and cross-linked collagen fibril paste.

3. The method as claimed in claim 2, wherein the cross-linked collagen fibril paste is prepared from the soluble collagen monomer solution by fibril reconstitution process and then treated with a cross-linking agent.

4. The method as claimed in claim 2, wherein the ratio of the collagen mass of the soluble collagen monomer solution to the cross-linked collagen fibril paste is in a range from 9.5:0.5 to 8:2.

5. The method as claimed in claim 1, wherein nine volumes of the collagen slurry are mixed with one volume of a phosphate buffer of pH 10˜12 and 0.1˜0.3 M to bring the slurry to physiological pH and ionic strength.

6. The method as claimed in claim 1, wherein the degassing of the collagen slurry is under vacuum of −400 to −700 mmHg.

7. The method as claimed in claim 1, wherein the collagen gel is formed at incubation temperature in the range of 30 to 40° C. under no agitation or shaking condition.

8. The method as claimed in claim 1, wherein the absorbent device comprises an absorbent material, a porous net covering the absorbent material, and a housing receiving the absorbent material and the porous net.

9. The method as claimed in claim 8, wherein the absorbent material is a sterile non-woven fabric pad, a sterile cotton pad, or the combination thereof.

10. The method as claimed in claim 8, wherein the porous net is a sterile nylon net, a sterile metal net, or the combination thereof.

11. The method as claimed in claim 1, wherein the collagen mat is flattened and dried under vacuum by the gel dryer at a heating temperature of 45±5° C.

12. A collagen membrane, which is prepared by a method comprising the following steps: preparing a collagen slurry; degassing the collagen slurry, and then forming collagen gel at a predetermined collagen concentration, ionic strength, pH value, and temperature; removing water in the collagen gel by an absorbent device to form a collagen mat; and flattening and drying the collagen mat under vacuum by a gel dryer.

13. The collagen membrane as claimed in claim 12, of which the density is in the range of 0.1 to 2 g/cm³.

14. The collagen membrane as claimed in claim 12, wherein collagen used in the collagen slurry is a soluble collagen monomer solution, or the combination of the soluble collagen monomer solution and a cross-linked collagen fibril paste.

15. The collagen membrane as claimed in claim 14, wherein the cross-linked collagen fibril paste is prepared from the soluble collagen monomer solution by fibril reconstitution process and then treated with a cross-linking agent.

16. The collagen membrane as claimed in claim 14, wherein the ratio of the collagen mass of the soluble collagen monomer solution to the cross-linked collagen fibril paste is in a range from 9.5:0.5 to 8:2.

17. The collagen membrane as claimed in claim 12, wherein nine volumes of the collagen slurry are mixed with one volume of a phosphate buffer of pH 10˜12 and 0.1˜0.3 M to bring the slurry to physiological pH and ionic strength.

18. The collagen membrane as claimed in claim 12, wherein the degassing is under vacuum of −400 to −700 mmHg.

19. The collagen membrane as claimed in claim 12, wherein the absorbent device comprises an absorbent material, a porous net covering the absorbent material, and a housing receiving the absorbent material and the porous net.

20. The collagen membrane as claimed in claim 19, wherein the absorbent material is a sterile non-woven fabric pad, a sterile cotton pad, or the combination thereof.

21. The collagen membrane as claimed in claim 19, wherein the porous net is a sterile nylon net, a sterile metal net, or the combination thereof.

22. The collagen membrane as claimed in claim 12, wherein the collagen gel is formed at incubation temperature in the range of 30 to 40° C. under no agitation or shaking condition.

23. The collagen membrane as claimed in claim 12, wherein the collagen mat is flattened and dried under vacuum by the gel dryer at a heating temperature of 45±5° C.
